(19) United States
(12) Patent Application Publication
(10) Pub. No.: US 2020/0129464 A1
(43) Pub. Date: Apr. 30, 2020

(54) Method and Composition for Increasing the Bioavailability of Carnitine

(71) Applicant: Lonza Inc., Morristown, NJ (US)

(72) Inventor: Aouatef Bellamine, Pennington, NJ (US)

(73) Assignee: Lonza Inc.

(21) Appl. No.: 16/665,774

(22) Filed: Oct. 28, 2019

Related U.S. Application Data
(60) Provisional application No. 62/751,866, filed on Oct. 29, 2018.

(51) Int. Cl. A61K 31/197; A61K 47/18

(52) U.S. Cl. CPC A61K 31/197; A61K 47/183

(57) ABSTRACT

A method and composition for improving the health of a mammal, such as by increasing the bioavailability of L-carnitine to the mammal. The method and composition includes administering an effective amount of branched chain amino acid metabolite substituted carnitine compound. The method and composition also includes administering an effective amount of branched chain amino acid metabolite substituted carnitine compound in order to improve the health of the mammal. The mammal's health may be improved by providing increased energy, anti-aging, anti-inflammatory and/or anti-oxidant properties, increased mitochondrial function, increased brain function, weight management and/or obesity management properties, exercise endurance and exercise recovery, increased protein synthesis, mTOR pathway activation, and/or Kreps cycle stimulation.

### BACKGROUND

L-carnitine was first discovered in 1905. Since then, extensive research has demonstrated the important role it plays in helping bodies utilize dietary fat for energy. Without sufficient L-carnitine in the body, humans and animals would not be able to utilize fat effectively and long-term health problems may occur.

In the body, L-carnitine is known to shuttle long-chain fatty acids across the inner-mitochondrial membrane so that the fatty acids can be metabolized and converted into energy. Without L-carnitine, these fatty acids would not be transported and properly utilized.

Mammals can obtain L-carnitine naturally from two sources. First, L-carnitine can be biosynthesized within the body. The body, however, can only produce small amounts of L-carnitine. Most of the daily requirements (75%) has to come from the diet or supplementation. L-carnitine can be obtained from meat-based foods, such as red meat.

In the past, L-carnitine and derivatives and salts thereof have been administered to mammals. For instance, L-carnitine and derivatives and/or salts thereof can be administered to a mammal when the diet of the mammal is low in L-carnitine. L-carnitine and derivatives and salts thereof have also been administered to mammals in order to maintain healthy weight, optimize energy, as an ergogenic aid during exercise, and to aid in recovery after exercise. Additionally, L-carnitine and salts and/or derivatives thereof have been added to infant formula, as infants lack the ability to produce L-carnitine, and many cow's milk and plant-derived formulas do not contain sufficient amounts of L-carnitine.

L-carnitine and derivatives and salts thereof have also been marketed to older mammals. As mammals increase in age, for instance, the availability of L-carnitine in the body decreases because food intake normally decreases and the body's ability to produce and use L-carnitine begins to drop. Supplementation with L-carnitine and derivatives and/or salts thereof has shown a positive influence on the aging process. Further, supplementation with L-carnitine and derivatives and/or salts thereof have been used to aid in building muscle and in muscle protein synthesis. Moreover, L-carnitine supplementation has also been found to be beneficial in improving cognitive function and fatigue Recently, L-carnitine-containing composition has been reported to improve skin ageing.

While L-carnitine supplements have provided great advances in improving health, muscle mass and function, and even brain health, further advances are needed. For example, in the past, to improve muscle mass in older mammals, amino acids such as leucine and organic acids like creatine have been administered along with L-carnitine to mammals in order to increase muscle mass and strength. In particular, many of the supplements administered to elderly mammals for increasing muscle protein synthesis comprise complicated and expensive mixtures of amino acids. For example, see U.S. Patent Application 2017/0173050, which discloses compositions containing carnitine, creatine and an amino acid for muscle protein synthesis. Also U.S. Pat. No. 7,790,688, which is incorporated by reference herein, discloses compositions containing a complex blend of essential amino acids, creatine, and low-glycemic carbohydrates. Further, it is well-known in the art that ingestion of 20 grams or more of essential amino acids is necessary for stimulation of muscle protein synthesis in the elderly. As protein intake increases, however, urea production significantly increases, requiring more frequent urinary excretion. Additionally, for muscle building and mood and/or cognitive function improvement, daily and/or high amounts of L-carnitine or a blend of L-carnitine and amino acids were needed.

Consequently, a need exists for a supplement that can increase the bioavailability of L-carnitine in a mammal. Additionally, it would be beneficial to provide a supplement that may increase the availability of branched chain fatty acids and/or branched chain amino acid and their derivatives and metabolites in a mammal. Further, it would be beneficial to provide a supplement that can increase muscle protein synthesis, lean mass, functional strength, and/or overall quality of life, in mammals without having any substantial adverse effects on other body functions. Additionally, it would be beneficial to provide a supplement that provides benefits in controlling weight, such as combating or preventing obesity, without adverse effects on the mammal's metabolism or body. Further, a composition that provides improvements in cognitive function and mood, without adverse effects on the mammal's metabolism or body, would be beneficial.

### SUMMARY

The present disclosure is generally directed to a method and composition for increasing the bioavailability of L-carnitine in a mammal. For instance, in one embodiment, the method comprises administering an effective amount of a branched chain amino acid metabolite substituted carnitine compound to a mammal, where the effective amount of the branched chain amino acid metabolite substituted carnitine compound is an amount sufficient to increase the bioavailability of L-carnitine in the mammal. In an additional embodiment, a branched chain amino acid metabolite substituted carnitine compound is a 2-methyl-butyrylcarnitine, isobutyrylcarnitine, 3-hydroxy-isovalerate carnitine, isovaleryl carnitine, 3-hydroxy isobutyryl carnitine or a mixture thereof. In a particular embodiment, the branched chain amino acid metabolite substituted carnitine compound is (S)-2-methyl-butyrylcarnitine and may be administered to the mammal as a mixture of (R) and (S)-2-methyl-butyrylcarnitine.

In a further embodiment, the increased bioavailability and the efficacy of L-carnitine is measured by an increase in an amount of L-carnitine in the mammal. Additionally, the increased amount of an L-carnitine metabolite may be measured in a tissue of a mammal, such as the muscle, brain, heart or skin tissues. In yet a further embodiment, the increased amount of an L-carnitine metabolite may be measured in a plasma of a mammal.

The method for increasing the bioavailability of L-carnitine in a mammal may also utilize an effective amount of branched chain amino acid metabolite substituted carnitine compound that is less than about 90% of an amount of L-carnitine or derivatives thereof needed to obtain the same amount of bioavailable L-carnitine in the same mammal. Further, the effective amount of a branched chain amino acid metabolite substituted carnitine compound may be from about 10% to about 50% of an amount of L-carnitine or derivatives thereof needed to obtain the same amount of bioavailable L-carnitine in the same mammal.

The present disclosure is also generally directed to a method for improving the health of a mammal. The method may include administering an effective amount of branched chain amino acid metabolite substituted carnitine compound to the mammal, where the effective amount of branched chain amino acid metabolite substituted carnitine compound is an amount sufficient to improve or increase at least one of: energy, anti-ageing effects, anti-inflammatory and/or anti-oxidant effects, mitochondrial function, brain function, muscle mass, muscle function, improved mobility, loss of fat content, improved triglycerides and cholesterol and/or weight management. In one embodiment, the health benefit is measured by mTOR expression, such as where the mammal has an mTOR expression that is at least about 10% greater than a period of time prior to administration of the effective amount of branched chain amino acid metabolite substituted carnitine compound.

In an additional embodiment, a branched chain fatty acid, may be administered with the effective amount of branched chain amino acid metabolite substituted carnitine compound. Further, the effective amount branched chain amino acid metabolite substituted carnitine compound is an amount sufficient to increase the metabolism of a branched chain fatty acid, and may improve exercise endurance, protein synthesis, and/or activate an mTOR pathway in the mammal, stimulate energy production in the Krebs cycle of the mammal, and/or improve weight management and obesity in the mammal. For instance, in one embodiment, the improvement in protein synthesis may be muscle protein synthesis.

The method for improving the health of a mammal may also utilize an effective amount of branched chain amino acid metabolite substituted carnitine compound that is less than about 90% of an amount of L-carnitine or derivatives thereof needed to obtain the same health improvement in the same mammal.

The present disclosure is also generally directed to a composition for improving the health of a mammal. The composition may include an effective amount of an L-carnitine moiety, wherein the L-carnitine moiety comprises at least about 40% or greater (S)-2-methyl-butyrylcarnitine by weight of the L-carnitine moiety. In an additional embodiment, the L-carnitine moiety contains up to about 20% (R)-2-methyl-butyrylcarnitine by weight of the L-carnitine moiety.

In one embodiment, the amount of branched chain amino acid metabolite substituted carnitine compound in the composition is sufficient to improve exercise endurance, exercise recovery, and/or protein synthesis, and/or activate an mTOR pathway in the mammal, stimulate energy production in the Krebs cycle of the mammal, and/or improve weight management and obesity in a mammal.

For instance, in one embodiment, the composition may contain an effective amount of branched chain amino acid metabolite substituted carnitine compound that is less than about 75% of an amount of L-carnitine or derivatives thereof needed to obtain the same improvement in the same mammal.

In yet a further embodiment, the present disclosure is generally directed a composition for improving the health of a mammal. In such an embodiment, the composition comprises an L-carnitine moiety and a branched chain fatty acid thereof, wherein the L-carnitine moiety comprises (R) or (S)-2-methybutyl-carnitine or a mixture of both.

Other features and aspects of the present disclosure are discussed in greater detail below.

### Definitions

The term “MET,” or “metabolic equivalent,” means the ratio of the rate of energy expended during an activity to the rate of energy expended at rest. A body at rest has a rate of energy expenditure of 1 MET. If a body performs a 2 MET activity, the body has expended 2 times the energy used by the body at rest.

The term “physical activity” means bodily movement with an energy expenditure rate equal to or greater than 3 MET. Non-limiting examples of “physical activity” include bicycling, sexual activity, giving birth, jogging, walking at a speed of about 3 mph or greater, calisthenics, jumping rope, running, sprinting, or any combinations thereof.

In one embodiment, “physical activity” can mean a negative energy balance in the mammal, such as weight loss, diets, aging, gestation, and lactation.

The term “physically active” means regularly participating in body movements with an energy expenditure rate of greater than or equal to 3 MET.

In one embodiment, “physically active” can mean regularly meeting medically recommended standards for amount, intensity, and type of physical activity performed by a mammal.

The terms “sedentary” or “sedentary activity” mean participating mainly or exclusively in body movements with an energy expenditure rate of less than 3 MET. Non-limiting examples of “sedentary” activities include sleeping, resting, sitting or reclining, watching television, writing, working at a desk, using a computer, typing, walking at a speed of less than about 3 mph, or any combinations thereof.

In one embodiment, “sedentary” can mean a failure to regularly meet medically recommended standards for amount, intensity, and type of physical activity performed by a mammal.

The term “L-carnitine” may contain L-carnitine and derivatives and/or salts thereof. L-carnitine can include L-carnitine base or derivatives and/or salts thereof including substantially pure crystalline L-carnitine, benzyl L-carnitine, L-carnitine HCL, L-carnitine L-tartrate, L-carnitine fumarate, propionyl L-carnitine, L-carnitine phosphate, acetyl L-carnitine L-aspartate, acetyl L-carnitine citrate, acetyl L-carnitine maleate, acetyl L-carnitine phosphate, acetyl L-carnitine fumarate, propionyl L-carnitine orotate, acetyl L-carnitine orotate, butyryl L-carnitine orotate, propionyl L-carnitine fumarate, L-carnitine oxalate, L-carnitine sulfate, GPLC glycine propionyl L-carnitine, and the like.

The term “branched chain amino acid or metabolites thereof” can mean amino acids such as leucine, isoleucine, and valine, as well as their metabolites. For instance, a metabolite of a branched chain amino acid may include 3-hydroxy-butanoyl-CoA, isovalerate, isobutyrate 3-hydroxy-isovaleryl-carnitine, 2-methyl-butyrate, 2-methyl-3-hydroxy-butyrate, propionyl-CoA, acetyl-CoA, succinyl-CoA, succinate, acetyl-coenzyme A, propionyl-coenzyme A, and succinyl-coenzyme A, as examples. Particularly, a metabolite of a branched chain amino acid may include any metabolite produced during the metabolism of a branched chain amino acid in a mammal.

The term “branched chain amino acid metabolite substituted carnitine compound”, means a branched fatty acid substituted carnitine, where the branched fatty acid forms an ester that is a metabolite of a branched amino acid compound which complexes with coenzyme A or carnitine. Examples of such carnitines include, for example any fatty acid derivatives thereof, particularly branched chain fatty acid derivatives, such as fatty acid derived from the metabolism of leucine, isoleucine, valine, dietary branched chain fatty acids, or combinations thereof, including the carnitine-esters of branched chain fatty acids and the branched chain fatty acid substrates thereof, such as 2-methylbutyrate, isobutyrate, isovalerate, 3-hydroxy-isovalerate, 3-hydroxy-butanoate, 3-hydroxy-isobutyrate, as well as, acetate, valerate, isovaleryl L-carnitine, L-leucyl L-carnitine, L-valyl L-carnitine, other L-amino acyl carnitines, salts of L-amino acyl L-carnitines.

The term “mammal” includes any mammal that may experience skeletal muscle degradation or synthesis, ageing effects on tissue such as skin, issues related to cognitive function and fatigue, weight gain or obesity, as well as deficiencies in energy level and/or metabolism, and includes human, canine, equine, feline, bovine, ovine, or porcine mammals.

The phrase “effective amount” means an amount of a compound that promotes, improves, stimulates, or encourages a response to the particular condition or disorder or the particular symptom of the condition or disorder.

The term “supplement” means a product in addition to the normal diet of the mammal but may be combined with a mammal's normal food or drink composition. The supplement may be in any form but not limited to a solid, liquid, gel, capsule, or powder. A supplement may also be administered simultaneously with or as a component of a food composition which may comprise a food product, a beverage, a pet food, a snack, or a treat. In one embodiment, the beverage may be an activity drink.

The term “functional strength” means an individual's ability to competently and safely perform daily life activities. In one embodiment, functional strength may be associated with energy, muscle potency, agility, flexibility, balance, mobility, joint health, and injury resistance.

The term “short chain fatty acid” means fatty acids, including branched chain fatty acids, having from 1 to 5 carbons.

The term “medium chain fatty acid” means fatty acids, including branched chain fatty acids, having from 6 to 12 carbons.

The term “long chain fatty acid” means fatty acids, including branched chain fatty acids, having 13 to 24 carbons.

### DETAILED DESCRIPTION

It is to be understood by one of ordinary skill in the art that the present discussion is a description of exemplary embodiments only, and is not intended as limiting the broader aspects of the present disclosure.

Generally speaking, the present disclosure is directed to a composition and method for increasing the bioavailability of L-carnitine, the bioavailability of a branched chain fatty acid, the bioavailability of a branched chain amino acid metabolite, or combinations thereof, in a mammal, as well as a composition and method for increasing the health of a mammal. The present disclosure has unexpectedly found that a composition that includes a branched chain fatty acid substrate that is chemically or enzymatically bound to carnitine, such as a branched chain amino acid metabolite substituted carnitine compound is effective in increasing the bioavailability of L-carnitine, the bioavailability of a branched chain fatty acid, a branched chain amino acid and/or derivatives and metabolites thereof, or combinations thereof, and improves the mammal's health, when administered to a mammal by an amount greater than would be expected based upon administration of the same amount of L-carnitine to the same mammal. Further, when an effective amount of the composition is administered to a mammal, the composition exhibits an unexpected synergism with branched chain fatty acids, including short, medium, and long branched chain fatty acids, and branched chain amino acids or metabolites thereof, as compared to the administration of the same amount of L-carnitine, providing health benefits to the mammal, among other advantages.

For instance, the present disclosure has unexpectedly found that when L-carnitine is enzymatically bound to a branched chain fatty acid substrate, the ester bond between the L-carnitine and the fatty acid has increased stability as compared to ester bonds between L-carnitine and unbranched fatty acids. Without intending to be bound by theory, it is believed that the branched chain of the fatty acid may decrease electron donation to the ester bond, minimizing the likelihood of dissociation of the bond, particularly in aqueous environments. Thus, the L-carnitine and the branched chain fatty acid do not dissociate as easily as a L-carnitine that is bound to an unbranched fatty acid. As the stability of the bond is improved, the branched chain fatty acid may be passively transported through the cell membrane when bound to the L-carnitine, instead of being restricted to active transport. Thus, the bioavailability of L-carnitine and branched chain fatty acids may be increased.

Particularly, in most circumstances, fatty acids are unable to diffuse through a cell membrane, even in their coenzyme-A form, and require a transporter or carrier. One such transporter or carrier is L-carnitine. However, most biological pathways utilize an active L-carnitine transporter, which may become saturated, resulting in any excess L-carnitine or fatty acid to be excreted by the bowel or metabolized by gut bacterial. Thus, in order to increase the bioavailability of L-carnitine and/or branched chain fatty acids in a cell, an additional transport system is needed. Accordingly, the present disclosure has unexpectedly found that due to the increased stability of the carnitine-ester-branched chain fatty acid bond, the branched chain fatty acid may be passively transported through the cell membrane, providing dual transportation of the fatty acid and carnitine into the cell, and increasing the bioavailability of the L-carnitine and branched chain fatty acid within the cell after transport.

Furthermore, in one embodiment, the branched chain fatty acid substrate may be a metabolite of the isoleucine pathway. While other branched chain fatty acid substrates may be used as defined above to increase the bioavailability of the respective branched chain fatty acids. When a metabolite of the isoleucine pathway is used as the branched chain fatty acid, succinate production, and thus, ATP production, may be increased in the mammal. Particularly, metabolites of the isoleucine pathway contribute to cellular energy in the form of ATP, as the metabolites are metabolized the entire way through to the Kreps cycle in the form of the Kreps cycle intermediate succinate. Thus, a composition that includes a branched chain fatty acid substrate that is enzymatically or chemically bound to L-carnitine, such as a composition that includes 2-methyl-butyrylcarnitine and the like, may increase the bioavailability of L-carnitine and/or the fatty acid metabolite, even when administered in amounts less than an amount of L-carnitine necessary to produce the same effect, may increase energy in the mammal, including mitochondrial function, and additionally, may improve anti-aging in the skin and muscles, including anti-aging of skin from within the skin cells themselves, anti-inflammatory and/or anti-oxidant effects, brain function, and improved weight management.

Particularly, as discussed above, a branched chain fatty acid substrate that is bound or substituted to L-carnitine, such as 2-methyl-butyrylcarnitine and the like, is a metabolite of isoleucine degradation, which is similar to the metabolites, isobutyryl-carnitine, the product of valine degradation, and isovaleryl-carnitine, the product of leucine degradation. In the branched chain amino acid metabolism pathway, 2-methyl-butyryl-CoA is produced as a metabolite, and is bound by L-carnitine for transport. After transport, the 2-methyl-butyrylcarnitine is metabolized to produce succinate for use in the Krebs cycle.

However, instead of beginning with isoleucine and L-carnitine as precursors, the present disclosure has surprisingly found that administering a branched chain fatty acid substrate that is enzymatically bound to carnitine, such as 2-methyl-butyrylcarnitine, to a mammal, results in unexpectedly higher amounts of biologically available L-carnitine in that mammal than would be expected based upon the metabolism of L-carnitine and isoleucine. However, while 2-methyl-butyrylcarnitine may be similar to the metabolites of valine and leucine degradation, due to isoleucine's metabolism pathway leading to the Kreps cycle, administration of a composition that includes 2-methyl-butyrylcarnitine may provide effects on cellular energy and availability as much as 10 times greater than the introduction of a leucine metabolite, which ends in the production of Hydroxy-methylbutyrate. Further, while 2-methyl-butyrylcarnitine is believed to be efficient and exhibit increased health benefits as compared to L-carnitine, it was also unexpectedly found that, as in many pathways, one of the enantiomers may be biologically active, providing more if not most of the benefit, for use in forming Kreps cycle intermediates and/or products such as succinate, precursors thereof, other intermediates known in the art, and ATP, and in the protein synthesis.

For instance, without intending to be bound by theory, the present disclosure has unexpectedly found that a composition that contains an amount of the (S) enantiomer of 2-methyl-butyrylcarnitine has a surprisingly greater effect and strength when administered to a mammal as compared to the same amount of L-carnitine, or the same amount of a mixture of the (R) and (S) enantiomers. Particularly, the present disclosure, without intending to be bound by theory, has found that the (R) enantiomer may produce metabolites that inhibit or slow down the (S) pathway, decreasing the succinate availability and the energy production in the Kreps cycle. Thus, while the present disclosure has found that a composition that includes both enantiomers of 2-methyl-butyrylcarnitine exhibits unexpectedly better results than compared to a composition that includes L-carnitine, a smaller amount yet of the (S) enantiomer may be more effective than a composition that includes a mixture of both enantiomers and/or L-carnitine separately, while still achieving the discussed benefits.

Particularly, the present disclosure has unexpectedly found that a composition according to the present disclosure is more efficacious per weight, such as per milligram (mg), administered than L-carnitine, and produces at least as strong of a response as L-carnitine, at the same or smaller doses. Moreover, in some instances, the present disclosure has surprisingly found that administration of only the (S) enantiomer of 2-methyl-butyrylcarnitine may result in a composition that is even more efficacious per milligram administered, than as compared to a composition that includes a mixture of (R) and (S) 2-methyl-butyrylcarnitine or to L-carnitine.

In one embodiment, the branched chain amino acid metabolite substituted carnitine compound may be administered in an amount which is less than about 90% of an amount of L-carnitine or derivatives thereof needed to obtain the same amount of bioavailable L-carnitine in the same mammal or to obtain the same health benefit in the same mammal, such as about 70% or less, such as about 65% or less, such as about 60% or less, such as about 55% or less, such as about 50% or less, such as about 40% or less, such as about 30% or less, such as about 25% or less than an amount of L-carnitine or derivatives thereof, or such as from about 20% to about 80%, or from about 25% to about 75%, such as from about 25% to about 50% of an amount of L-carnitine or derivatives thereof needed to obtain the same amount of bioavailable L-carnitine in the same mammal or to obtain the same health benefit in the same mammal. Additionally or alternatively, the branched chain amino acid metabolite substituted carnitine compound, such as 2-methyl-butyrylcarnitine may be administered in an amount of from about 0.75 times or less of an amount of L-carnitine or derivatives thereof needed to obtain the same amount of bioavailable L-carnitine in the same mammal or to obtain the same health benefit in the same mammal, such as about 0.8 or less, such as about 0.7 times or less, such as about 0.65 times or less, such as about 0.6 times or less, such as about 0.55 times or less, such as about 0.5 times or less, such as about 0.4 times or less, such as about 0.3 times or less, such as about 0.25 times or less than an amount of L-carnitine or derivatives thereof, or such as from about 0.25 times to about 0.85 times, or from about 0.35 times to about 0.75 times, such as from about 0.4 times to about 0.5 times of an amount of L-carnitine or derivatives thereof needed to obtain the same amount of bioavailable L-carnitine in the same mammal or to obtain the same health benefit in the same mammal, e.g. in some embodiments, an amount of L-carnitine or derivatives thereof needed to obtain the same amount of bioavailable L-carnitine in the same mammal or to obtain the same health benefit in the same mammal may be greater, such as from about 1.25 to about 4 times greater, as compared to an amount of the 2-methyl-butyrylcarnitine sufficient to elicit the same response in the same mammal.

Particularly, in one embodiment using the above percentages, the amount of 2-methyl-butyrylcarnitine needed to obtain the same amount of bioavailable L-carnitine in the same mammal may only be in reference to an amount of the (S) enantiomer of 2-methyl-butyrylcarnitine administered to the mammal even though the (S)-2-methyl-butyrylcarnitine may be administered alone or as part of a mixture of (R) and (S) enantiomers, or the amount may be in reference to an amount of the mixture of the (R) and (S) enantiomers. In a further embodiment, only about 25% of an amount of the (S) enantiomer, administered alone or as part of a mixture of the (R) and (S) enantiomers, as compared to an amount of L-carnitine, may be needed to obtain the same amount of bioavailable L-carnitine in the same mammal or to obtain the same health benefit in the same mammal.

For instance, in one embodiment, the L-carnitine moiety may include a racemic mixture of the (R) and (S) enantiomers. Alternatively, the (S) enantiomer may be present in the L-carnitine moiety in an amount of from about 0.1% to about 100% by weight, such as about 10% to about 90%, such as from about 25% to about 75% by weight of the L-carnitine moiety, or such as an amount of from about 5% to about 50% or greater, such as from about 60% or greater, such as from about 75% or greater, such as from about 90% or greater, such as about 100% or less, such as about 95% or less, such as about 85% or less, such as about 70% or less, such as about 55% or less, such as about 45% or less, such as about 25% or less by weight of the L-carnitine moiety.

Additionally, the (R) enantiomer may be present in the L-carnitine moiety in an amount of from about 0.1% to about 50% by weight, such as about 10% to about 40%, such as from about 15% to about 35% by weight of the L-carnitine moiety, or such as an amount of from about 5% or greater, such as from about 10% or greater, such as from about 15% or greater, such as from about 20% or greater, such as from about 25% or greater, such as from about 35% or greater, such as from about 50% or greater, such as from about 60% or greater, such as from about 75% or greater, such as from about 90% or greater, such as about 95% or less, such as about 85% or less, such as about 70% or less, such as about 50% or less, such as about 45% or less, such as about 25% or less by weight of the L-carnitine moiety.

While a composition has been discussed that includes only the (S) enantiomer of 2-methyl-butyrylcarnitine or a mixture of the (R) and (S) enantiomers of 2-methyl-butyrylcarnitine, it should be understood that regardless of the composition used, the (S) enantiomer may be obtained by separation of the (S) enantiomer from the racemic mixture of the (R) and (S) enantiomers or by formation of the (S) enantiomer separately. Particularly, in one embodiment, all or a portion of the (R) enantiomer may be removed, leaving a composition of 2-methyl-butyrylcarnitine that is all, or at least greater than 50%, (S) enantiomer. Of course, alternatively, the (S) enantiomer may be removed from the 2-methyl-butyrylcarnitine composition, and is use to form a composition of the L-carnitine moiety that is completely, or at least predominantly, (S) 2-methyl-butyrylcarnitine. In yet a further embodiment, the (S) enantiomer of 2-methyl-butyrylcarnitine may be formed separately from the 2-methyl-butyrylcarnitine racemic mixture, and is thus formed as only the (S) enantiomer instead of being separated from the racemic mixture. Thus, when used herein, a composition of 2-methyl-butyrylcarnitine that contains all of a portion of the (S) enantiomer, may be referring to a (S) enantiomer produced or separated according to any of the embodiments discussed above, unless noted otherwise.

In a further embodiment, the composition may include a branched chain fatty acid, or metabolite thereof, administered in conjunction with the L-carnitine moiety. As discussed above, the present disclosure has unexpectedly found that 2-methyl-butyrylcarnitine exhibits an unexpected synergism with branched chain fatty acids as compared to the effects exhibited in respect to the administration of L-carnitine.

In an embodiment the composition may include a branched chain amino acid, such as a branched chain amino acid and/or metabolites thereof, the branched chain amino acid may comprise leucine, isoleucine, valine, and/or metabolites thereof. Of course, the present method and composition may include administration of branched chain fatty acids in conjunction with the L-carnitine moiety, such as in a single or integrated supplement, or the L-carnitine moiety may be administered separately in an effective amount to synergistically interact with branched chain fatty acids already being taken in by the mammal, such as branched chain fatty acids that the mammal is obtaining from its diet or from existing supplementation. In an embodiment where the branched chain fatty acids are administered as part of the composition of the present disclosure, the branched chain fatty acids may be administered at the same time as the L-carnitine moiety, or before, or after the L-carnitine moiety. In a further embodiment, the branched chain amino acids and L-carnitine moiety may be contained in a single supplement as defined above for administration to the mammal.

Particularly, while the composition according to the present disclosure may include various additives as may be known in the art, which will be discussed in greater detail herein, as well as the branched chain fatty acid, in one embodiment, L-carnitine moiety may be present in the composition at a concentration greater than about 5% by mass, such as greater than about 10% by mass, such as greater than about 15% by mass, such as greater than about 20% by mass, such as greater than about 25% by mass, such as greater than about 30% by mass, such as greater than about 35% by mass, such as greater than about 40% by mass, such as greater than about 45% by mass, such as greater than about 50% by mass, such as greater than about 55% by mass, such as greater than about 60% by mass, such as greater than about 65% by mass, such as greater than about 70% by mass, such as greater than about 75% by mass. In general, L-carnitine moiety may be present in the composition at a concentration up to about 100% by mass, but may also be present in the composition in an amount such as less than about 75% by mass, such as less than about 65% by mass, such as less than about 60% by mass, such as less than about 55% by mass, such as less than about 50% by mass, such as less than about 45% by mass, such as less than about 40% by mass, such as less than about 35% by mass, such as less than about 30% by mass, such as less than about 25% by mass, such as less than about 20% by mass, such as less than about 15% by mass, such as less than about 10% by mass.

As stated above, the composition may comprise the L-carnitine moiety in combination with a branched chain amino acid or metabolite thereof. The branched chain amino acid may be leucine, isoleucine, valine, and any derivatives, metabolites, and/or salts thereof. Leucine, for instance, is known to stimulate protein synthesis via association with eukaryotic initiation factors in translation. In one embodiment, the amino acid may comprise a metabolite of leucine, such as HMB.

In one embodiment, the at least one branched chain amino acid may be present in the composition at a concentration greater than about 10% by mass, such as greater than about 15% by mass, such as greater than about 20% by mass, such as greater than about 25% by mass, such as greater than about 30% by mass, such as greater than about 35% by mass, such as greater than about 40% by mass, such as greater than about 45% by mass, such as greater than about 50% by mass, such as greater than about 55% by mass, such as greater than about 60% by mass, such as greater than about 65% by mass, such as greater than about 70% by mass, such as greater than about 75% by mass. In general, the branched chain fatty acid or metabolite thereof may be present in the composition at a concentration less than about 75% by mass, such as less than about 70% by mass, such as less than about 65% by mass, such as less than about 60% by mass, such as less than about 55% by mass, such as less than about 50% by mass, such as less than about 45% by mass, such as less than about 40% by mass, such as less than about 35% by mass, such as less than about 30% by mass, such as less than about 25% by mass, such as less than about 20% by mass, such as less than about 15% by mass.

In one embodiment, the branched chain fatty acid includes a mixture of one or more branched chain fatty acids, derivatives, and/or metabolites thereof. In such an embodiment, the entire mixture of branched chain fatty acids or metabolites thereof is contained in the composition according to the above concentrations. However, in a further embodiment, only a single branched chain fatty acid is used as the branched chain fatty acid component of the composition. In such an embodiment, the single branched chain fatty acid may be present in the same amounts as discussed above in regards to the concentration of all of the branched chain fatty acids in the composition.

In one embodiment, the branched chain fatty acid(s), derivatives, and/or metabolites thereof may be included in the composition as free form organic compounds. Alternately, the branched chain fatty acid(s) and/or metabolites thereof may be included in the composition as intact proteins and/or other macromolecules. In a further embodiment, the branched chain fatty acid(s), derivatives, and/or metabolites thereof may be included in the composition as a combination of free form organic compounds and intact protein and/or other macromolecules.

The different components can be present in the composition at various ratios depending upon the particular application and the desired result. The weight ratio between the L-carnitine moiety and the amino acid, for instance, can generally be from about 1:100 to about 100:1, such as from about 1:3 to about 3:1. In one embodiment, the weight ratio between the amino acid derivative and the amino acid may be from about 2:1 to about 1:2.

In a further embodiment, the composition may also comprise one or more vitamins. In one particular embodiment, the composition may comprise vitamin D3. Vitamin D can regulate muscle contractility. Other vitamins may include but are not limited to vitamin A, vitamin B1, vitamin B2, vitamin B3, vitamin B6, vitamin B9, vitamin B12, vitamin C, vitamin E, vitamin K, riboflavin, niacin, folic acid, pyridoxine, thiamine, pantothenic acid, biotin, and any combinations thereof. The composition may further comprise other minerals, herbs, botanicals, and essential fatty acids. In one embodiment, the composition may comprise magnesium and/or salts thereof.

In one embodiment, the one or more vitamins may be present in the composition in an amount of about 1 to about 5,000 IU per dose, such as about 10 to about 2,500 IU per dose, such as about 50 to about 1,500 IU per dose, such as about 100 IU to about 1,000 IU per dose, such as about 250 IU to about 750 IU per dose, such as about 300 IU to about 600 IU per dose.

The present disclosure is also directed to methods of administering the composition disclosed herein in order to increase the bioavailability of L-carnitine and/or a metabolite of a branched chain fatty acid in the mammal and/or to provide a health benefit to the mammal. Thus, administration of the L-carnitine moiety, optionally in combination with a branched chain fatty acid or instead administered to a mammal who may be deficient in branched chain fatty acids, or that is not deficient in branched chain fatty acids, such as a mammal that is currently undergoing supplementation or that has or is obtaining sufficient branched chain fatty acids from its diet, has been discovered to improve the health of the mammal, such as by providing increased energy, anti-aging, anti-inflammatory and/or anti-oxidant properties, increased mitochondrial function, increased brain function, weight management and/or obesity management properties, improved joint health, improved skin health, particularly skin health from within via improved cellular function, exercise endurance and exercise recovery, increased protein synthesis, mTOR pathway activation, and/or Kreps cycle stimulation. Moreover, the above advantages and benefits may be realized without any adverse consequences. In addition, in one embodiment, the advantages and benefits may be realized when administering an amount of the composition that is less than an amount of L-carnitine needed to elicit the same response in the mammal.

Regardless of whether a branched chain fatty acid is administered with the L-carnitine moiety, the L-carnitine may increase the bioavailability of an, or at least one, metabolite of a branched chain fatty acid. For instance, the present disclosure has unexpectedly found that by administering an L-carnitine moiety, branched chain fatty acid metabolite metabolism may be unexpectedly increased. Particularly, the present inventors have found that by administering an amount of an L-carnitine moiety, metabolites of branched chain amino acids, such as those metabolites produced during oxidative metabolism of the branched chain amino acids, may be increased as compared with the levels expected based upon the administration of L-carnitine. For example, an increase in the bioavailability of the branched chain amino acid metabolite may be measured by an increase in a Kreps cycle intermediate and/or a Kreps cycle product, particularly, a Krebs cycle intermediate that may be measured may be succinate or a precursor thereof and/or an end product such as ATP and increased cellular energy.

In one embodiment, a mammal is administered an effective amount of the L-carnitine moiety, such as an amount sufficient to increase the bioavailability of L-carnitine or to provide one or more of the above discussed benefits. The L-carnitine moiety may be administered in a dosage from about 5 to 10,000 milligrams per day, such as from about 5 to about 5,000 milligrams per day, such as from about 50 milligrams to about 3,000 milligrams per day. The dosage, for instance, can be greater than about 100 milligrams per day, such as greater than about 250 milligrams per day, such as greater than about 500 milligrams per day, such as greater than about 750 milligrams per day. Based on body mass, the dosage can be from about 1 milligram per kilogram of body weight per day to about 1,000 milligrams per kilogram body weight per day. For example, the dosage may be from about 5 milligrams per kilogram body weight per day to about 750 milligrams per kilogram body weight per day. In one particular embodiment, the dosage can be from about 10 milligrams per kilogram body weight per day to about 500 milligrams per kilogram body weight per day. In another particular embodiment, the dosage can be greater than about 1 milligrams per kilogram body weight per day, greater than about 5 milligrams per kilogram body weight per day, greater than about 10 milligrams per kilogram body weight per day, greater than about 15 milligrams per kilogram body weight per day, greater than about 20 milligrams per kilogram body weight per day, greater than about 25 milligrams per kilogram body weight per day, greater than about 30 milligrams per kilogram body weight per day, or greater than about 35 milligrams per kilogram body weight per day.

In a further embodiment, the mammal can be administered an effective amount of only the (S) enantiomer of 2-methyl-butyrylcarnitine. In such an embodiment, the (S)-2-methyl-butyrylcarnitine may be administered in a dosage from about 1 to about 5,000 milligrams per day, such as from about 5 milligrams to about 1,500 milligrams per day, such as from about 50 milligrams to about 1000 milligrams per day, such as from about 100 milligrams to about 750 milligrams per day. The dosage, for instance, can be greater than about 10 milligrams per day, such as greater than about 50 milligrams per day, such as greater than about 75 milligrams per day, such as greater than about 100 milligrams per day, such as greater than about 150 milligrams per day, such as greater than about 250 milligrams per day, such as greater than about 500 milligrams per day, and/or less than about 5000 milligrams per day, such as less than about 2500 milligrams per day, such as less than about 1000 milligrams per day, such as less than about 750 milligrams per day. Based on body mass, the dosage can be from about 1 milligram per kilogram of body weight per day to about 500 milligrams per kilogram body weight per day. For example, the dosage may be from about 5 milligrams per kilogram body weight per day to about 400 milligrams per kilogram body weight per day. In one particular embodiment, the dosage can be from about 10 milligrams per kilogram body weight per day to about 250 milligrams per kilogram body weight per day. In another particular embodiment, the dosage can be greater than about 1 milligrams per kilogram body weight per day, greater than about 5 milligrams per kilogram body weight per day, greater than about 10 milligrams per kilogram body weight per day, greater than about 15 milligrams per kilogram body weight per day, greater than about 20 milligrams per kilogram body weight per day, greater than about 24 milligrams per kilogram body weight per day, greater than about 28 milligrams per kilogram body weight per day, or greater than about 30 milligrams per kilogram body weight per day, and/or less than about 500 milligrams per kilogram body weight per day, such as less than about 400 milligrams per kilogram body weight per day, such as less than about 300 milligrams per kilogram body weight per day, such as less than about 250 milligrams per kilogram body weight per day. Of course, in an embodiment where the amount of the (S) enantiomer alone is measured for administration of the composition, the composition may still include a mixture of (R) and (S) enantiomers as discussed above, however, the amount administered is based upon the amount of the (S) enantiomer present in the composition.

The composition can be administered regularly, such as at least two to four times a week. For instance, the composition may be administered to the mammal at least every one to three days. Further, the composition may be administered more than one time per day. For instance, the composition may be administered to the mammal one to four times per day. In one particular embodiment, the composition is administered daily. The dosage when using a mixture of the (R) and (S) enantiomers of the 2-methyl-butyrylcarnitine can be from about 5 to 30,000 milligrams per day, such as from about 5 to about 20,000 milligrams per day, such as from about 10 milligrams to about 10,000 milligrams per day, such as from about 20 milligrams to about 5,000 milligrams per day, such as from about 50 milligrams to about 2,500 milligrams per day. Based on body mass, the dosage can be from about 1 milligram per kilogram of body weight per day to about 10,000 milligrams per kilogram body weight per day. For example, the dosage may be from about 5 milligrams per kilogram body weight per day to about 7,500 milligrams per kilogram body weight per day, such as from about 10 milligrams per kilogram body weight per day to about 5,000 milligrams per kilogram body weight per day, such as from about 15 milligrams per kilogram body weight per day to about 2,500 milligrams per kilogram body weight per day, such as from about 20 milligrams per kilogram body weight per day to about 1,000 milligrams per kilogram body weight per day, such as from about 25 milligrams per kilogram body weight per day to about 750 milligrams per kilogram body weight per day, such as from about 30 milligrams per kilogram body weight per day to about 500 milligrams per kilogram body weight per day, such as from about 35 milligrams per kilogram body weight per day to about 250 milligrams per kilogram body weight per day.

In an alternative embodiment, the dosage when administering only (S)-2-methyl-butyrylcarnitine can be from about 5 to 15,000 milligrams per day, such as from about 5 to about 10,000 milligrams per day, such as from about 10 milligrams to about 5,000 milligrams per day, such as from about 20 milligrams to about 2,500 milligrams per day, such as from about 50 milligrams to about 1,250 milligrams per day, such as from about 50 milligrams to about 1,000 milligrams per day. Based on body mass, the dosage can be from about 1 milligram per kilogram of body weight per day to about 5,000 milligrams per kilogram body weight per day. For example, the dosage may be from about 5 milligrams per kilogram body weight per day to about 3,750 milligrams per kilogram body weight per day, such as from about 10 milligrams per kilogram body weight per day to about 2,500 milligrams per kilogram body weight per day, such as from about 15 milligrams per kilogram body weight per day to about 1,250 milligrams per kilogram body weight per day, such as from about 20 milligrams per kilogram body weight per day to about 500 milligrams per kilogram body weight per day, such as from about 25 milligrams per kilogram body weight per day to about 375 milligrams per kilogram body weight per day, such as from about 30 milligrams per kilogram body weight per day to about 250 milligrams per kilogram body weight per day, such as from about 35 milligrams per kilogram body weight per day to about 125 milligrams per kilogram body weight per day. Of course, in an embodiment where the amount of the (S) enantiomer alone is measured for administration of the composition, the composition may still include a mixture of (R) and (S) enantiomers as discussed above, however, the amount administered is based upon the amount of the (S) enantiomer present in the composition.

The composition can be administered to the mammal in any suitable form using any suitable administration route. For example, the composition can be administered orally alone, in combination with a food composition, or as part of a food or nutraceutical composition. The composition of the present disclosure can be a nutritional supplement in the form of a pill, tablet or capsule. Alternatively, the composition may be incorporated into a food or beverage. For instance, the composition may be incorporated in to a milkshake drink, a juice, a cereal bar, a vitamin such as a gummy vitamin, or a powder. The powder can be mix with any suitable liquid for ingestion.

The composition can be administered orally as a solid, liquid, suspension, or gas. The composition may be administered via buccal or sublingual administration. In one embodiment, the composition may be administered as a capsule, tablet, caplet, pill, troche, drop, lozenge, powder, granule, syrup, tea, drink, thin film, seed, paste, herb, botanical, and the like.

In addition to being administered orally, the supplement dose can also be administered using other routes including intranasal, intravenous, intramuscular, intragastric, and the like.

When the composition is combined with a food or beverage composition, the food or beverage composition may comprise any suitable composition for consumption by the mammal. Such compositions include complete foods or beverages intended to supply the necessary dietary requirements for mammal or food supplements such as treats and snacks. The food composition may comprise pellets, a drink, a bar, a prepared food contained in a can, a milk shake drink, a juice, a dairy food product, or any other functional food composition. The food composition may also comprise any form of a supplement such as a pill, soft gel, gummy figurine, wafer, or the like.

The mammal treated in accordance with the present disclosure can comprise any suitable mammal. For instance, the mammal may be human or canine. The composition can be fed to a mammal of any age such as from parturition through the adult life in the mammal. In various embodiments the mammal may be a human, dog, a cat, a horse, a pig, a sheep, or a cow. In many embodiments, the mammal can be in early to late adulthood. For instance, the active mammal may have an age that is at least 10%, such as least 15%, such as least 20%, such as least 25%, such as least 30%, such as least 35%, such as least 40%, such as least 45%, such as least 50%, such as least 55%, such as least 60%, such as least 65%, such as least 70%, such as least 75%, such as least 85%, such as least 90%, such as least 95% of its expected life span. The mammal may have an age such that it is less than about 95%, such as less than about 90%, such as less than about 85%, such as less than about 80%, such as less than about 75%, such as less than about 70%, such as less than about 65%, such as less than about 60%, such as less than about 55%, such as less than about 50%, such as less than about 45%, such as less than about 40%, such as less than about 35%, such as less than about 30%, such as less than about 25%, such as less than about 20%, such as less than about 15%, such as less than about 10% of its expected life span. A determination of life span may be based on actuarial tables, calculations, or the like.

The composition may be administered to the mammal according to the present disclosure regardless of the frequency, intensity, or type of physical activity performed by the mammal. The mammal may participate in physical activities with various MET values. In one embodiment, the mammal may regularly participate in light to intense physical activity. Light physical activity may have a MET of from about 3 MET to about 6 MET. Moderate physical activity may have a MET of from about 6 MET to about 10 MET. Intense physical activity may have a MET of about 10 MET or greater. In another embodiment, the mammal may infrequently participate in physical activity. In yet another embodiment, the mammal may lead a sedentary lifestyle, wherein the mammal may rarely or never participate in physical activity. In a sedentary lifestyle, a mammal may participate mainly or exclusively in sedentary activities.

The composition may be administered to the mammal before, during, or after a period of physical activity. Alternately, the composition may be administered to the mammal before, during, or after a period of sedentary activity. For instance, the composition may be administered to the mammal during an extended period of bed rest or other extended period of inactivity. In a further embodiment, the composition may exhibit the discussed effects without requiring the mammal to undergo or to have not undergone any particularly activity.

As discussed above, administration of the 2-methyl-butyrylcarnitine may provide a health benefit to the mammal greater than a benefit when the same mammal is administered the same amount of L-carnitine, or as compared to a mammal that has not received any L-carnitine supplementation.

In one embodiment, the health benefit improved may be increased muscle protein synthesis, which may be determined by monitoring the biomarker, mTOR, in skeletal muscle. Specifically, mTOR expression can be determined and recorded before and after a period of activity. For a mammal treated in accordance with the present disclosure, mTOR expression and activation before and after a period of time may vary by more than 10%, such as by more than 20%, such as by more than 40%, such as by more than 60%, such as by more than 80%, such as by more than 100%, such as by more than 150%, such as by more than 200%.

In one embodiment, the mammals treated in accordance with the present disclosure may have total mTOR values after a period of activity that are at least 10%, such as at least 20%, such as at least 30%, such as at least 40%, such as at least 50%, such as at least 60%, such as at least 100% greater than the same mammal that is not administered the composition or as compared to a mammal that is administered the same amount of L-carnitine.

In addition to increased muscle protein synthesis, the composition can also increase functional strength. In particular, the composition can increase lean muscle mass and upper and lower body strength. Functional strength can be measured by a composite endpoint of strength and muscle measures. The composite endpoint may be the product of the values for muscle mass (kg), upper extremity strength by dynamometer (kg), lower extremity strength by dynamometer (kg), and 6-minute walk test (meters). Comparative measurements can be taken prior to and after a period of activity.

In one embodiment, mammals treated in accordance with the present disclosure may have composite endpoints after a certain period of time that are at least 10%, such as at least 25%, such as at least 50%, such as at least 75%, such as at least 100% more than the same mammal that is not administered the composition or as compared to a mammal that is administered the same amount of L-carnitine.

The composition of the present disclosure may also reduce inflammation. Inflammation, in one embodiment, can be determined by monitoring one or more biomarkers, such as, for example only TNF-α, in skeletal muscle or the plasma. TNF-α is an inflammatory marker involved in protein degradation. Reduction in TNF-α values may indicate a reduction in protein degradation and muscle wasting. TNF-α values can be determined and recorded before and after a period of activity. For a mammal treated in accordance with the present disclosure, TNF-α values before and after a period of time may vary by more than 0.5%, such as by more than 1%, such as by more than 5%, such as by more than 10%, such as by more than 20%, such as by more than 40%, such as by more than 60%, such as by more than 80%, such as by more than 100%, such as by more than 150%, such as by more than 200%. Of course, as may be known in the art, many other biomarkers may be measured to determine a reduction in inflammation, and may display a reduction in inflammation in one or more body tissues or plasmas.

In one embodiment, the mammals treated in accordance with the present disclosure may have total TNF-α values after a period of activity that are at least 0.5%, such as at least 1%, such as at least 5%, such as at least 10%, such as at least 20%, such as at least 30%, such as at least 40%, such as at least 50%, such as at least 60%, such as at least 100% less than the same mammal that is not administered the composition or as compared to a mammal that is administered the same amount of L-carnitine.

In another embodiment, the improvement in health may be an improvement in energy levels of the mammal.

In yet a further embodiment, the improvement in health may be anti-ageing effects on the mammal, such as anti-aging of the skin and/or muscles (sarcopenia) of a mammal.

Additionally or alternatively, the improvement in health may be improved or more efficient mitochondrial function.

Furthermore, the improvement in health may be improved brain function. In one embodiment, the composition may contribute to improved cognitive function, alertness, and memory.

Moreover, the improvement in health may be improved weight management. For instance, the composition according to the present disclosure may contribute to an increase in muscle mass, an increase in muscle function, and a loss of fat content. In a particular embodiment, the improvement may be in managing obesity.

Additionally, the composition may provide a health benefit such as improved exercise endurance and recovery.

Moreover, as previously discussed, the L-carnitine moiety may also work synergistically with other dietary ingredients, such as branched chain fatty acids in the composition or already present in the mammal to achieve the above health benefits or to further improve the above benefits due to the increased bioavailability of the L-carnitine and increased cellular energy.

In addition to the discussion above in regards to muscle protein synthesis and mTOR activation, the L-carnitine moiety and the branched chain amino acid may work synergistically to enhance new protein synthesis, in one embodiment, the synthesis may be in muscle cells.

Moreover, the L-carnitine moiety may interact synergistically with the branched chain amino acid to stimulate energy production by the Kreps cycle.

Furthermore, in addition to the weight management benefits discussed above, it was unexpectedly found that, while the L-carnitine moiety provides health benefits related to weight management, the combination of a L-carnitine moiety and a branched chain amino acid may exhibit an unexpected synergism to provide weight management and obesity control greater than would be expected based upon an additive effect of the L-carnitine moiety or branched chain amino acid administered alone.

In one embodiment, the compositions of the present disclosure may also contain other amino acids, including but not limited to alanine, arginine, asparagine, aspartate, cysteine, glutamic acid, glutamine, glycine, proline, serine, tyrosine, histidine, isoleucine, lysine, methionine, phenylalanine, threonine, tryptophan, valine, and any combinations thereof.

The composition of the present disclosure may further comprise one or more excipients as further additives in the composition in conjunction with the 2-methyl-butyrylcarnitine. Exemplary but non-limiting excipients and/or additives include antiadherents, such as magnesium stearate; binders, such as saccharides, sugar alcohols, gelatin, and synthetic polymers; coatings, such as cellulose ether hydroxypropyl methylcellulose (HPMC), shellac, corn protein zein, gelatin, fatty acids, and waxes; coloring agents, such as titanium oxide and azo dyes; disintegrants, such as modified starch sodium starch glycolate and crosslinked polymers including polyvinylpyrrolidone and sodium carboxymethyl cellulose; fillers, such as maltodextrin; flavoring agents, such as mint, liquorice, anise, vanilla, and fruit flavors including peach, banana, grape, strawberry, blueberry, raspberry, and mixed berry; glidants, such as fumed silica, talc, and magnesium carbonate; lubricants, such as talc, silica, and fats including vegetable stearin, magnesium stearate, and stearic acid; preservatives, such as antioxidants, vitamins, retinyl palmitate, selenium, the amino acids cysteine and methionine, citric acid, sodium citrate, and parabens; sorbents; sweeteners, such as sucrose and sucralose; and vehicles, such as petrolatum and mineral oil.

In one embodiment, the composition of the present disclosure may be combined with various additives and components that can improve one or more properties of the composition. For example, in one embodiment, the additive composition may be combined with a stabilizer package that may serve to stabilize at least one property of the composition. In one particular embodiment, for instance, a stabilizer package may be added to the composition in an amount sufficient to reduce the hydroscopic properties of the composition and/or prevent the composition from absorbing moisture. A stabilizer package may also be combined with the composition in order to improve the handling properties of the composition. For instance, the stabilizer package may allow the composition to have better flow properties, especially when in granular form.

In one embodiment, the composition may be combined with a polymer binder in conjunction with a stabilizer package. In addition, a coating material may also be applied to the composition after the composition has been combined with the polymer binder and the stabilizer package. The coating material, for instance, may contain at least one fat. In accordance with the present disclosure, the above components can be added to any suitable pharmaceutical composition in addition to the composition of the present disclosure. For instance, the above components may be added to any pharmaceutical composition containing a carnitine or an amino acid.

The polymer binder and the stabilizer package may be combined with the composition in a manner that homogeneously incorporates the stabilizer package into the product. In one embodiment, for instance, the composition of the present disclosure is first combined with a polymer binder, such as through a spray dry process, and then combined with the stabilizer package. The polymer binder may comprise any suitable pharmaceutically acceptable polymer, such as film-forming polymers and/or polysaccharides. Particular examples of polymer binders that may be used in accordance with the present disclosure include starch, maltodextrin, gum arabic, arabinogalactan, gelatin, and mixtures thereof. In one embodiment, the polymer binder is added to the pharmaceutical composition in an amount of at least about 5% by weight, such as at least about 8% by weight, such as at least about 10% by weight, such as at least about 15% by weight. One or more polymer binders are present in the composition in an amount less than about 50% by weight, such as in an amount less than about 45% by weight, such as in an amount less than about 40% by weight, such as in an amount less than about 35% by weight, such as in an amount less than about 30% by weight.

In one embodiment, the polymer binder may comprise a starch, such as a modified starch. The starch, for instance, may be derived from corn or waxy maize. In one embodiment, the starch may comprise HI-CAP100 starch sold by National Starch and Chemical Company.

In an alternative embodiment, the polymer binder may comprise arabinogalactan. Arabinogalactan is a soluble polysaccharide that not only can serve as a polymer binder but may also provide other benefits. For instance, arabinogalactan may enhance the adaptive immune response in some circumstances. Arabinogalactan is described, for instance, in U.S. Pat. No. 8,784,844, which is incorporated herein by reference.

In one embodiment, larch arabinogalactan may be used as the polymer binder. Larch arabinogalactan is a highly branched polysaccharide that is composed of galactose units and arabinose units in the approximate ratio of 6:1. Larch arabinogalactan is extracted from large trees. The polysaccharide has a galactan backbone with side chains of galactose and arabinose. Arabinogalactan is commercially available from Lonza Ltd.

Once the polymer binder is combined with the composition such as through a spray dry process, the resulting mixture can then be combined with a stabilizer package. In one embodiment, the stabilizer package comprises oxide particles in combination with a salt of a carboxylic acid. In one particular embodiment, the stabilizer package may comprise a dry product, such as a powder or granular product that is combined with the composition and polymer binder. The combination of oxide particles and a salt of a carboxylic acid have been found to provide numerous advantages and benefits when combined with the composition. For instance, the stabilizer package has been found to stabilize the composition and make the composition less hydroscopic. The composition is also easier to handle and, when in granular form, produces a free-flowing product.

The oxide particles that may be added to the pharmaceutical composition may comprise silica. For instance, the oxide particles may comprise precipitated silica particles. The silica particles may have a particle size (d50, laser defraction following ISO Test 13320) of less than about 55 microns, such as less than about 40 microns, such as less than about 30 microns, such as less than about 25 microns, such as less than about 20 microns, such as less than about 15 microns, such as less than about 12 microns, such as less than about 10 microns, such as less than about 8 microns, such as less than about 6 microns, such as less than about 4 microns, such as less than about 2 microns, such as less than about 1 micron. The particle size is typically greater than about 0.5 microns, such as greater than about 1 micron. The particles may have a specific surface area (ISO Test 9277) of greater than about 120 m2/g, such as greater than about 130 m2/g, such as greater than about 150 m2/g, such as greater than about 170 m2/g, such as greater than about 200 m2/g, such as greater than about 220 m2/g. The specific surface area is generally less than about 500 m2/g. The oxide particles, such as the silica particles, can be present in the pharmaceutical composition in an amount greater than about 0.01% by weight, such as in an amount greater than about 0.05% by weight, such as in an amount greater than about 0.1% by weight. The oxide particles are generally present in an amount less than 5% by weight, such as in an amount less than about 2% by weight, such as in an amount less than about 1.5% by weight, such as in an amount less than 0.5% by weight.

In addition to the oxide particles, the stabilizer package may also include a salt of a carboxylic acid. The salt of a carboxylic acid may comprise a salt of a fatty acid. The fatty acid, for instance, may have a carbon chain length of from about 6 carbon atoms to about 40 carbon atoms, such as from about 12 carbon atoms to about 28 carbon atoms. In one embodiment, the salt of the carboxylic acid may comprise a stearate salt. The stearate salts that may be used include calcium stearate, sodium stearate, magnesium stearate, mixtures thereof, and the like. In one embodiment, the salts of the carboxylic acid may include both hydrophilic groups and hydrophobic groups. The salt of the carboxylic acid may be present in the composition in an amount greater than about 0.5% by weight, such as in an amount greater than about 1% by weight, such as in an amount greater than about 1.5% by weight. The salt of the carboxylic acid is generally present in an amount less than about 5% by weight, such as in an amount less than about 4% by weight, such as in an amount less than about 3% by weight.

In addition to the polymer binder and the stabilizer package, the composition may include various other components and ingredients. In one embodiment, for instance, the composition may contain a citric acid ester, such as a citric acid ester of a mono and/or diglyceride of a fatty acid. The composition may also contain a lecithin, such as a lecithin obtained from rapeseed, sunflower, and the like. The above components can be present in the composition in relatively minor amounts, such as less than about 2% by weight, such as less than about 1.5% by weight, such as less than about 1% by weight. The above components are generally present in an amount greater than about 0.05% by weight, such as in an amount greater than about 0.1% by weight.

Once the above components are combined together to form the composition, the composition can optionally be combined with a coating material. In one embodiment, for instance, the composition may comprise a granular composition to which a coating material is applied that contains a fat. The coating material, for instance, may comprise a hydrogenated oil, such as hydrogenated palm oil. In one particular embodiment, the coating material may comprise hydrogenated palm oil combined with palm stearine. In one embodiment, the hydrogenated oil may be present in the pharmaceutical composition in an amount from about 5% to about 35% by weight. The palm stearine, on the other hand, may be present in the pharmaceutical composition in an amount from about 2% to about 10% by weight. When present together, the weight ratio between the hydrogenated palm oil and the palm stearine may be from about 10:1 to about 1:1, such as from about 6:1 to about 2:1. In one embodiment, the hydrogenated palm oil and the palm stearine are present at a weight ratio of about 4:1.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A method for increasing bioavailability of L-carnitine and/or a branched chain amino acid metabolite in a mammal, the method comprising:
providing a branched chain amino acid metabolite substituted carnitine compound; administering an effective amount of the branched chain amino acid metabolite substituted carnitine compound to the mammal,
wherein the effective amount of branched chain amino acid metabolite substituted carnitine compound is an amount sufficient to increase the bioavailability of L-carnitine, bioavailability of the branched chain amino acid metabolite, or both the bioavailability of L-carnitine and the bioavailability of the branched chain amino acid metabolite in the mammal.

2. The method of claim 1, wherein the branched chain amino acid metabolite substituted carnitine compound comprises 2-methyl-butyrylcarnitine, isobutyrylcarnitine, 3-hydroxy-isovalerate carnitine, isovaleryl carnitine, 3-hydroxy isobutyryl carnitine or a mixture thereof.

3. The method of claim 1, wherein the increased bioavailability of L-carnitine is measured by an increase in an amount of an L-carnitine in the mammal.

4. The method of claim 3, wherein the increased amount of an L-carnitine is measured in a tissue of a mammal, in a plasma of a mammal, in the brain of a mammal, the skin of a mammal or the heart of a mammal.

5. The method of claim 4, wherein the tissue is muscle tissue.

6. The method of claim 1, wherein the increase in the bioavailability of the branched chain amino acid metabolite is measured by an increase in a Kreps cycle substrate, product, or combinations thereof.

7. The method of claim 6, wherein the Kreps cycle substrate is succinate or a precursor thereof.

8. The method of claim 6, wherein the Kreps cycle product is an increase in ATP or an increase in the production of energy.

9. The method of claim 1, wherein the effective amount of the branched chain amino acid metabolite substituted carnitine compound is less than about 90% of an amount of L-carnitine or derivatives thereof needed to obtain the same amount of bioavailable L-carnitine in the same mammal.

10. The method according to claim 2, wherein the branched chain amino acid metabolite substituted carnitine compound comprises 2-methyl-butyrylcarnitine.

11. The method according to claim 10, wherein the 2-methyl-butyrylcarnitine comprises (S)-2-methyl-butyrylcarnitine.

12. The method of claim 11, wherein the (S)-2-methyl-butyrylcarnitine is administered to the mammal as a mixture of (R) and (S)-2-methyl-butyrylcarnitine.

13. A method for improving health of a mammal, the method comprising: administering an effective amount of branched chain amino acid metabolite substituted carnitine compound
wherein the effective amount of (branched chain amino acid metabolite substituted carnitine compound is an amount sufficient to improve or increase at least one of: energy, anti-ageing effects, anti-inflammatory and/or anti-oxidant effects, mitochondrial function, brain function, muscle mass, muscle function, loss of fat content, improvement in joint health, improvement in skin health, weight management, other health benefits known to L-carnitine, or a combination thereof.

14. The method of claim 13, wherein the mammal has an mTOR expression that is at least about 10% greater than a period of time prior to administration of the effective amount of branched chain amino acid metabolite substituted carnitine compound.

15. The method of claim 13, further comprising administering a branched chain amino acid or metabolite thereof with the effective amount of branched chain amino acid metabolite substituted carnitine compound.

16. The method of claim 13, wherein the effective amount of branched chain amino acid metabolite substituted carnitine compound is administered with an amount of the at least one branched chain amino acid or metabolite thereof, sufficient to improve exercise endurance, exercise recovery, and/or protein synthesis, and/or activates a mTOR pathway in the mammal, stimulate energy production in the Krebs cycle of the mammal, and/or improve weight management and obesity in the mammal.

17. The method of claim 16, wherein the protein synthesis is muscle protein synthesis.

18. The method of claim 13, wherein the effective amount of branched chain amino acid metabolite substituted carnitine compound is less than about 90% of an amount of L-carnitine, or derivatives thereof, needed to obtain the same health improvement in the same mammal.

19. The method of claim 13, wherein the branched chain amino acid metabolite substituted carnitine compound comprises 2-methyl-butyrylcarnitine, isobutyrylcarnitine, 3-hydroxy-isovalerate carnitine, isovaleryl carnitine, 3-hydroxy isobutyryl carnitine or a mixture thereof.

20. The method according to claim 13, wherein the branched chain amino acid metabolite substituted carnitine comprises (S)-2-methyl-butyrylcarnitine

21. A composition for improving health of a mammal, wherein the composition comprises an effective amount of a L-carnitine moiety, and wherein the L-carnitine moiety comprises at least about 80% or greater (S)-2-methyl-butyrylcarnitine by weight of the L-carnitine moiety.

22. The composition of claim 21, wherein the composition further includes at least one branched chain fatty acid metabolite thereof.

23. The composition of claim 21, wherein the amount of (S)-2-methyl-butyrylcarnitine is sufficient to improve exercise endurance, exercise recovery, and/or protein synthesis, and/or activate a mTOR pathway in the mammal, stimulate energy production in the Krebs cycle of the mammal, and/or improve weight management and obesity in a mammal

24. The composition of claim 21, wherein the effective amount of (S)-2-methyl-butyrylcarnitine is less than about 75% of an amount of L-carnitine, or derivatives thereof, needed to obtain the same improvement in the same mammal.

25. The composition of claim 21, wherein the L-carnitine moiety contains up to about 20% of (R)-2-methyl-butyrylcarnitine by weight of the L-carnitine moiety.
